(19) United States
(12) Patent Application Publication
(10) Pub. No.: US 2010/0297213 A1
(43) Pub. Date: Nov. 25, 2010

(54) Method For Making Patches By Electrospray

(76) Inventors: Bertrand Dupont, Aix en Provence (FR); Ludovic Tatoulian, Paris (FR); Pascale Ehouarn, Gif Sur Yvette (FR)

Correspondence Address: JONES DAY, 222 EAST 41ST ST, NEW YORK, NY, 10017, US

(21) Appl. No.: 12/863,981

(22) PCT Filed: Jan. 23, 2009
(86) PCT No.: PCT/FR09/50094

§ 371 Date: Jul. 21, 2010

(30) Foreign Application Priority Data
Jan. 23, 2008 (FR) 0850406

(51) Int. Cl. B05D 3/14; A61K 9/70; A61K 38/02

(52) U.S. Cl. 424/447; 427/2.31; 514/1.1

(57) ABSTRACT

The invention relates to patches and methods for manufacturing patches intended for skin application of a substance wherein the patch includes a conductive support, and a liquid formulation of the substance is deposited on the support of the patch by electrohydrodynamic spraying.

The present invention generally relates to the manufacturing of patches intended for cutaneous application of substances. The invention more particularly relates to methods and devices for manufacturing such patches by ElectroHydroDynamic Spraying (EHDS). The invention is applicable to the manufacturing of any type of patch, which may notably be used for pharmaceutical, cosmetic, vaccinal and/or diagnostic applications, in humans or animals.

### TECHNOLOGICAL BACKGROUND OF THE INVENTION

Cutaneous application of a substance by means of a patch has many applications in human or animal health. Indeed, it may allow development of efficient diagnostic tests or methods for transferring active ingredients to the skin. Even if the human epidermis forms a barrier against the entry into the body of external agents, the skin is not a perfect seal. Several investigations have experimentally shown the feasibility of such methods under various conditions. Further, several systems of patches are presently marketed in the field of detection of allergies.

Applying substances on the skin has many advantages as compared with other methods of administration, such as injection, and notably absence of any risk of contamination, absence of pain, handling ease, or further the possibility for the patient of himself/herself administering the substance.

Different types of patch have been described in the literature. Patches intended for local action such as for example plasters, patches, bandages, or cupules, may notably be mentioned.

Other patches have been described, intended for general action, i.e. transdermal patch systems. In this type of patch, the substance may be delivered to the organism, either through passive diffusion or through diffusion facilitated by a physico-chemical process (iontophoresis, electroporation, sonophoresis), or further by mechanical action (micro-needles).

In the case of dermal patches with passive diffusion, a substance is typically deposited on a surface of the patch (called a support) and placed in contact with the skin. The patch may include an occlusive chamber or a condensation compartment. Application of the patch on the skin enables contact between the substance and the skin and diffusion of the substance into the layers of the epidermis or into the organism.

Whichever the type of patch used, it is important to have efficient, reproducible and industrializable methods for preparing them. Thus for example, the electrostatic patch described above is typically prepared according to a manufacturing method using so-called <<duster>> systems, as the one shown in document WO 07/122226. This method consists of applying on a patch support, the biologically active substance as a dry powder, by means of a rotary roller (or propeller) which during its rotary travel, recovers powder and applies it against the support. Nevertheless, this manufacturing system generates losses of powder and therefore of substance, additionally because of problems of deposition outside the patch support (for example on the perimeter of the patch) and/or of clogging of the powders on the walls of the deposition reactor, in particular when the powders have a very fine grain size or when the powder particles have a particular shape (for example powders obtained by freeze-drying). These losses on the one hand force the use of significant amounts of substance powder, which increases the manufacturing cost of the patch, but also generates difficulties for controlling the amounts of active substance deposited on the patch and the homogeneity of the deposit.

Patent application US 2005/220853 relates to a medical article including an adhesive substrate and a therapeutic agent deposited on this substrate. Different deposition techniques are mentioned, but this document does not describe how to obtain homogeneous and controlled deposit under industrial conditions on the support of a patch.

Application WO 03/094811 relates to a method for manufacturing a bandage intended for treating wounds. This bandage, which may be directly made on a wound or beforehand on a support, is obtained by depositing fibers which do not have any biochemical function. Moreover, this document does not describe how to obtain a substance deposit (active ingredient), under industrial and pharmaceutical conditions, on the support of a patch.

There is therefore a need in the prior art for improved methods for producing patches containing a biological substance. In particular, when dealing with dry patches which use the natural loss of water of the skin for solubilizing on the skin the substance to be administered, a deposit as hydrophilic as possible is needed in order to obtain rapid and complete dissolution as soon as the patch is laid on the skin.

### SUMMARY OF THE INVENTION

The present invention is directed to providing an improved method for industrial manufacturing of patches, and in particular of dry patches, by using the ElectroSpray technique (or ElectroHydroDynamic Spraying or further <<EHDS>>).

With the method according to the invention, it is possible to control the size, the electric charge and the production frequency of the droplets produced by EHDS from a liquid formulation and starting from this, it is possible to control the size and the frequency of the substance particles projected on the support of the patch, in order to obtain a homogeneous deposit and control the amount of substance deposited on the patch. The charged particles follow the lines of the electric field between the nozzle and the support, which more or less allows accurate localization of the location of the deposit on the support by controlling the field lines.

Thus, the object of the invention is a method for manufacturing a patch intended for cutaneous application of a substance, the method comprising electrohydrodynamic spraying deposition of a liquid formulation of the substance on the support of the patch.

Another object of the invention relates to a method for manufacturing a patch intended for cutaneous application of a substance, characterized in that the patch includes a conductive support and in that the method comprises deposition of the substance on the support of the patch by electrohydrodynamic spraying of a liquid formulation of the substance.

In a preferred embodiment, the substance is dissolved in a solvent in order to form the formulation before spraying, for example an aqueous solvent possibly comprising a surfactant.

In another preferred embodiment, the substance or the liquid formulation is directly sprayed as droplets having an average diameter of less than or equal to about 20 μm, preferably 5 μm, more preferentially 1 μm.

In another preferred embodiment, the formulation is sprayed at a rate comprised between 0.1 and 1.5 mL/hour.

In another preferred embodiment, spraying is achieved at a voltage comprised between 1 and 10 kvolts.

In another preferred embodiment, the method comprises a step for treating, preferably by heating, the support, during or after spraying, in order to obtain a deposit in the form of dry residues or to reduce the humidity rate of the achieved deposit.

A particular object of the invention lies in a method for manufacturing a patch comprising a support coated with a substance, characterized in that it comprises deposition by electrohydrodynamic spraying of the substance on the support, according to the following steps:

a) placing a conductive support at a distance from a spraying nozzle;

b) providing the substance as a liquid to the spraying nozzle;

c) submitting the substance to an electric field so as to form an aerosol between the nozzle and the support; and

d) collecting the formed aerosol on the support.

As indicated, the substance is in liquid form (liquid formulation) and it is therefore the liquid formulation which is subject to an electric field in step c).

The method comprises an optional additional step for forming and/or packaging the support for forming a patch.

The object of the invention is also a patch intended for cutaneous application of a substance, which may be obtained by the manufacturing method described above.

Another object of the invention lies in a patch comprising a conductive support.

The object of the invention is further an installation of a device for manufacturing a patch, characterized in that it comprises at least one electrohydrodynamic spraying device (preferably comprising at least one spray nozzle (11) and at least one counter-electrode and/or contact with the ground positioned so as to generate an electric field and form an aerosol from a formulation (21) and means for feeding the installation with patch conductive supports (31). In a particular embodiment, the device comprises several spray nozzles (11) which either operate simultaneously or not, each nozzle creating a substance deposit on a patch support. The different nozzles are advantageously mounted on an insulating support.

The method according to the invention is particularly advantageous for manufacturing patches, in particular dry patches, since it notably ensures:
homogeneity of the deposit over the whole surface area of the patch which has to be covered with substance, which is particularly advantageous for administration through the skin of the patient,
accurate control of the dose deposited on each patch in order to in particular meet regulatory pharmaceutical constraints,
structure and quality of the deposit in each patch in order to obtain a substance deposit as bioavailable as possible (e.g., solubilization of the deposit after laying the patch on the skin and by perspiration).

The invention further describes means for reducing as much as possible the time for depositing the substance and for achieving in parallel and simultaneously several deposits on the same machine, which is required for application of EHDS technology for applying a substance on a patch at industrial production rates.

The invention is adapted to any type of substance, notably active substances, such as antigens, allergens, or drugs, and to any type of patch, i.e. any device which may be applied on a skin area of a subject in order to put it into contact with a substance or to create a moisturizing area. These may be patches with passive, facilitated or mechanical diffusion, adhesive patches, bandages, plasters, cupules or (trans)dermal patches. A dermal device with passive diffusion of the occlusive type or with a condensation compartment is advantageously used.

### CAPTIONS OF THE FIGURES

FIG. 1 illustrates a patch during manufacturing according to an embodiment of the method according to the invention.

FIG. 2 is a sectional view of an exemplary patch structure.

FIG. 3 illustrates a patch with a conductive support.

FIG. 4 illustrates a principle of a wide strip for manufacturing patches by ElectroSpray.

FIG. 5 illustrates a comparison of operating voltage and liquid flow rate ranges obtained with peanut formulations with and without ethanol.

FIG. 6 illustrates an exemplary deposit made on a PET/GOLD film with focusing of the aerosol by the polarized shielding ring and polarization of the insulating washer of the patch.

FIG. 7 illustrates SEM photographs of a deposit of dried peanut particles made on a polymeric film covered with aluminium.

FIG. 8 illustrates SEM photographs of deposits of porous peanut layers made on a polymeric film PET covered with gold.

FIG. 9 illustrates a profile (made between the centre and the edge of the deposit) of the elementary composition of porous peanut layer deposits made on a polymeric film PET covered with gold.

FIG. 10 illustrates operating voltage and liquid flow rate ranges with/without a ring connected to the ground; D_{ext nozzle}/D_{int nozzle} (mm)−4/0.3; D_{int ring} (mm)=20; Dᵢ₋ₑ=D_{nozzle-ring}=20 mm; D_{ring-plane}=20 mm.

### DETAILED DESCRIPTION OF THE INVENTION

The invention relates to an improved industrial method for producing patches, to an installation or a device for its application, as well as to novel patches having advantageous and usable properties in any mammal in pharmaceutical, cosmetic or diagnostic applications, for example. The invention is notably based on a step for electrohydrodynamically spraying a liquid formulation forming or containing a substance of interest, in order to deposit said substance of interest on a conductive support adapted to the making of patches.

The invention, for the first time, allows deposition of a substance on the support of a patch by electrohydrodynamic spraying. As this emerges from experimental examples conducted by the inventors, this method provides control of the size, the charge and the frequency of the particles projected on the support of the patch, and a homogeneous deposit may be obtained and the amount of deposited substance on the patch may be controlled.

ElectroSpray, or electrohydrodynamic spraying (<<EHDS>>) is a method used for producing substances often in a dry form and in very small amounts, for example for analyzing substances in spectroscopy, making micro-deposits of substance for diagnosis, coating of surfaces with active substances, producing micro- and nano-particles, or further producing microfibres (see notably WO 99/49981, WO2006/010845, U.S. Pat. No. 7,259,109, U.S. Pat. No. 5,349,186, FR 1 288 034, FR 1 087 802).

However, although the principle of EHDS is known in various applications, transposition of this technique to the industrial manufacturing of patches has never been considered or made possible. In particular, EHDS includes certain limitations and technical constraints which may appear incompatible with pharmaceutical and industrial use, which requires rapidity, robustness and biocompatibility. Among these constraints, mention may notably be made of:
the low throughput of produced substances, synonym of low yield,
high sensitivity of the method towards outer conditions and perturbations, and
quasi-impossibility for certain formulations and for certain required flow rates, of temporarily interrupting the aerosol and taking it up again without damaging the quality of the deposit.

The present invention now shows that EHDS may be adapted to the industrial and controlled manufacturing of patches. The present invention also follows from the development of optimum conditions under which the spraying method may be applied for manufacturing patches.

Thus, a particular object of the invention lies in a method for manufacturing a patch comprising a support coated with a substance, characterized in that it comprises the deposition of the substance (21) on the support (31), by electrohydrodynamic spraying, according to the following steps:

a) placing a conductive support (31) at a distance from a spray nozzle (11);

b) providing the substance in a liquid formulation (21) to the spraying nozzle (11);

c) submitting the formulation (21) to an electric field so as to form an aerosol (22) between the nozzle (11) and the support (31); and

d) collecting the particles from the aerosol (22) which are formed on the support (31).

The present application now shows that it is possible to obtain by EHDS, homogeneous and reproducible deposits, under conditions compatible with industrial and pharmaceutical use. The present application also describes the optimum conditions under which this method may be applied, and notably the liquid formulation of the sprayed substance, the voltage used, the flow rate used, the geometry of the electrodes, in order to obtain regular and homogeneous deposits.

According to the ElectroSpray principle, in a stable production mode, polarization of the nozzle (11) induces separation of the electric charges borne by the ions present in the liquid. Under the action of the electric field at the outlet of the nozzle (11), the positive and negative charges within the liquid separate and those of same polarity as the nozzle (11) migrate towards the surface of the liquid; the liquid is polarized. The electric charges with a polarity opposite to the applied potential are at the nozzle-liquid interface while a portion of the charges of same polarity are at the surface of the liquid. If the electric field at the surface of the liquid increases sufficiently, the electric pressure normal to the surface of the liquid, directed towards the inside of the drop, increases. For certain voltage and liquid flow rate conditions, the electric and hydrodynamic pressures are in equilibrium at the surface of the liquid: there is electrohydrodynamic equilibrium. In this case, the drop at the outlet of the nozzle assumes the shape of a stable liquid cone (Taylor cone) at the end of which emerges a liquid jet. A hydrodynamic instability propagates along the jet which is fragmented into highly charged micronic drops. Electrostatic repulsions between the charged drops create a radial extension effect inducing the formation of an aerosol (22) and thus promote homogeneity of the projection while preventing any inter-particle agglomeration or coagulation.

Depending on the adjustment of the parameters of the method, such as in a non-exhaustive way, the liquid flow rate, the voltage and polarity of the nozzle, and depending on the intrinsic properties of the liquid (21) such as in a non-exhaustive way, electric conductivity, dynamic viscosity, surface tension, specific gravity and relative permittivity, the invention in particular shows that it is possible to industrially obtain from the control of the frequency and of the diameter of the droplets forming the aerosol (22), a deposit of particles with a controlled diameter or a controlled deposit of layered substances.

### Geometry of the Electrodes

The electric field is formed by the voltage applied between the liquid at the outlet of the nozzle and one or more electrodes, with any combination of the following counter-electrodes: a counter-electrode (16) polarized or connected to the ground, the support being positioned between the nozzle and the counter-electrode (16), a ring-shaped counter-electrode (12) or a plate with holes, either polarized or connected to the ground, positioned between the spray nozzle and the support, and one or more contacts (41, 44) connected to the ground and to the contact of the support (31).

According to a particular embodiment, the electric field is formed by applying a potential difference between the spray nozzle (11) and the support (31), the latter being connected to the ground.

According to another embodiment, the electric field is formed by applying a potential difference between the spray nozzle (11) and a ring-shaped counter-electrode (12) or plate with holes, polarized or connected to the ground, positioned between the spray nozzle and the support.

In a particular embodiment, the electric field is formed by applying a potential difference between the liquid formulation at the end of the spray nozzle (21) via this spray nozzle (11), and the ring-shaped counter-electrode (12) (also designated as a shielding ring), either polarized or connected to the ground.

According to another embodiment, the electric field is formed by applying a potential difference between the spray nozzle (11) and one or more contacts (41, 44) connected to the ground and to the contact of the support (31).

In a particularly peculiar embodiment, the spray device comprises a counter-electrode (12) (also designated as a shielding ring) (FIG. 6). This shielding ring (12) is in conductive material, notably in a metal material. It may have a conducting portion and an insulating portion.

The shielding ring (12) is preferably in the form of a metal ring or a plate with holes, positioned perpendicularly to the spraying direction of the formulation, preferably at a distance comprised between 0 and 30 millimetres from the nozzle (11). With the shielding ring (12) thereby crossed by the aerosol (22) projected on the support of the patch (31) it is possible to guarantee the stability of the method. It may be connected to the ground or to a high voltage generator.

Generally, the shielding ring has the following advantages:

i) Possibility of controlling the diameter of the deposits. Indeed, the applied potential on the ring allows the intensity of the electrostatic repulsions to be controlled between the polarized ring and the charged drops of the same polarity as the ring. In which case, the higher the potential applied to the ring, the more electrostatic repulsions increase between the ring and the drops. Accordingly, the area of the support covered by the flow of drops, therefore the diameter of the deposit, decrease gradually as the applied potential on the ring increases;

ii) Increase the robustness of the method by shielding the production area, i.e. by stabilizing the production of the aerosol, no longer between the nozzle and the support but between the nozzle and the shielding ring. In such a case, and for optimum dimensions, shapes and position of the ring, the production of the aerosol is quasi independent of what is happening outside the area located between the nozzle and the ring. This stability requirement is an actual necessity in the case of industrial manufacturing of patches in order to make the method sufficiently robust, to avoid destabilization and thus obtain a sufficient effective production time.

### Spray Nozzle

According to the embodiments, the spray nozzle may be totally conducting or totally insulating, or have a conducting portion and an insulating portion. It forms, when it is conductive and connected to a high voltage power supply (13), an electrode with which the formulation (21) may be polarized. When it is insulating, it is the support of the nozzle, in direct contact with the liquid to be polarized, which is then conductive and connected to the high voltage. The nozzle (11) typically has an orifice of circular shape for letting through the liquid formulation (21), the outer diameter of which is advantageously comprised between 0.05 and 8 millimetres and the inner diameter of which is advantageously comprised between 0.05 and 1 millimetre.

According to an embodiment, the device may comprise several spray nozzles (11) and the formulation (21) is sprayed by several nozzles (11).

Indeed, one of the drawbacks of the electrospray method is the low liquid flow rate delivered by a nozzle (order of magnitude (OM): 0.1-100 mL/h), inducing low production yield. In order to optimize the method of the invention, a system including several nozzles has been developed, which allows an increase in the number of patches produced per unit time by as much. Such a system was made by the inventors, in spite of the technical difficulties related to predictable interference problems between electric fields and electrostatic edge effects.

Indeed, the electric fields required for EHDS have to be similar from one nozzle to the other and notably not be subject to edge effects favourable to spatial modifications of the electric field. In addition to the geometrical electric field E_{g} allowing the formation of the cone and of the liquid jet, the electric field of the space charge consisting of the charged particles induces coulombian repulsions between the latter advantageously preventing inter-particle coagulation but may, on the one hand, perturb the stabilization of the method by interacting with the neighbouring sprays, have an influence on the direction of the aerosols on the other hand. Now, the latter should be perpendicular to the surface of the support on the one hand and for industrial production, be parallel to each other on the other hand.

Setting up a battery of several nozzles comes up against electrostatic edge effects, the nozzles located on the edges producing tilted sprays and therefore unusable for production. This drawback may theoretically be limited by installing, at the end of each row of nozzles, a system producing an electric field similar to the one which is produced by the adjacent spray, which will counterbalance it.

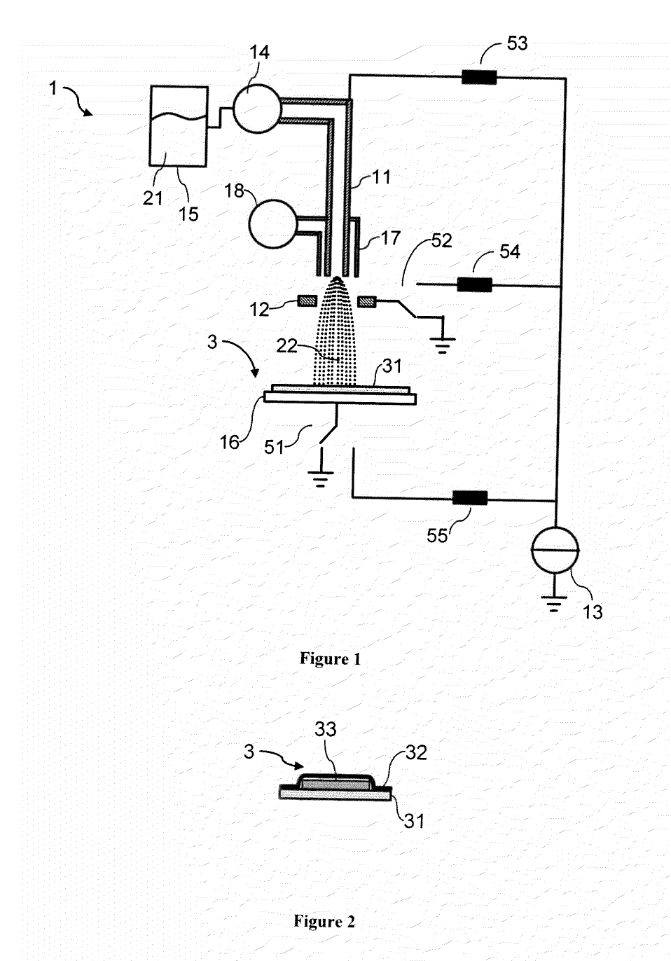

We have shown that it is possible to avoid these additional systems, by mounting the nozzles on an insulating support: upon initially starting the method, during an initial stabilization phase, a strong interaction between the liquid cones is observed which induces tilting of the latter for the two nozzles on the ends (FIG. 1.a). This angle decreases after a few seconds and the sprays then become quasi vertical (FIG. 1.b) and remain so. Without being certain of the explanation put forward, it is likely that this effect is due to equilibration of the polarization of the insulators which surrounds the nozzle and in particular its support.

Tests were carried out on a method for depositing peanut extract with a system of 3 nozzles (D_{ext/int nozzle}=4/0.3 mm) inserted on an insulating PVC support. An adequate distance between the 37 mm nozzles, in a nozzles-plane configuration and at a liquid flow rate of 1 mL/h per nozzle enabled stabilization of the method and the active ingredient was able to be deposited under similar conditions for the three nozzles on a support, for which the surface was 15 mm.

In a preferred embodiment, the method of the invention comprises simultaneous spraying from several nozzles, preferably from 2-10 nozzles. More preferably, the nozzles used are mounted on an insulating support.

### High Voltage Power Supply

The electric field required for forming the aerosol (22) is generated by using a high DC voltage power supply.

The electrospray device thus advantageously comprises a positive or negative high DC voltage power supply (13), applying a potential difference between the nozzle (11) and the support, and/or the counter-electrode, and/or the shielding ring for the whole duration of the production of the patches (21). The power supply (13) typically provides a current from −5 to +5 microamperes and applies a DC voltage from −30 to +30 kilovolts.

In a particularly preferred embodiment, the method is applied under a voltage comprised between 1 and 10 kvolts.

### Liquid Formulation

As indicated, the substance in liquid form is used in the method. The nature of this liquid formulation may be adapted in order to improve the performances of the method. In particular, the inventors have shown that the electric conductivity and viscosity of this formulation may be controlled and in certain cases, adapted in order to obtain the best industrial performances of the method.

Thus, the substance is preferably dissolved in a solvent. The amount of dissolved substance depends on its solubility.

The solvent may be any solvent compatible with pharmaceutical use, preferably an organic solvent, capable of solubilizing the substance of interest.

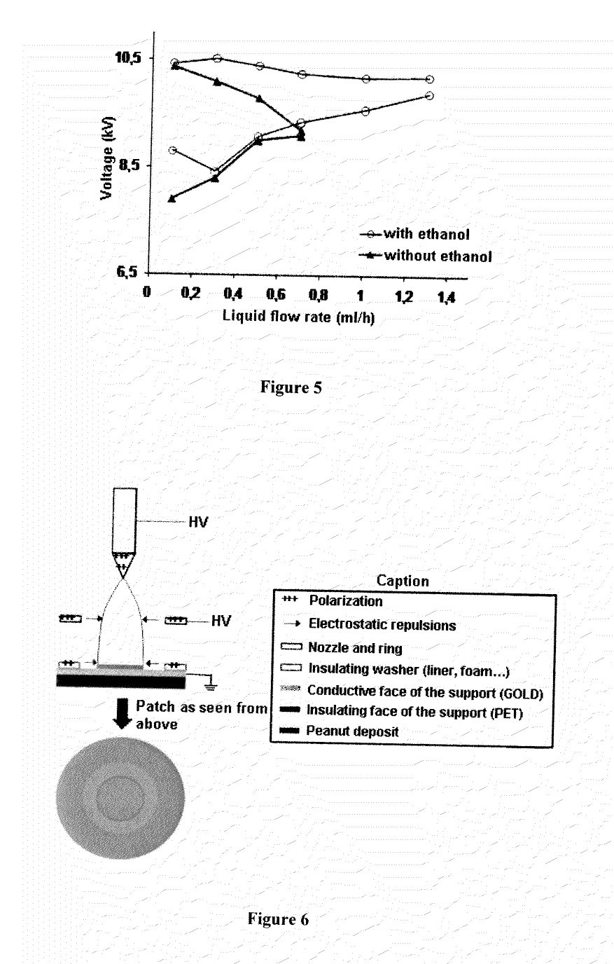

The solvent used during the method for dissolving the substance and thereby forming the liquid formulation, may be selected according to the properties of the substance and according to the drying rate or quality which is desirably obtained. For example, the solvent may be water, with which it is possible to avoid deterioration of certain substances during the production of the patches. Nevertheless, in order to accelerate evaporation of the solvent, it may then be advantageous to add an alcohol to the aqueous formulation, for example ethanol. In a particular embodiment, the liquid formulation is therefore an aqueous solvent comprising from 1-15% (by total volume of the solution), preferably from 1-10% (by total volume of the solution) of alcohol, preferably ethanol. The obtained results show that such a formulation is particularly adapted to mixtures of proteins, such as allergens. Moreover, the obtained results also show that the use of ethanol allows improvement in the stability of the method, as illustrated in FIG. 5.

In a preferred embodiment, the liquid formulation comprises the substance dissolved in an aqueous solvent comprising 1-10% by volume of ethanol. This type of formulation is particularly adapted for polypeptides (e.g., proteins) and peptides.

Moreover, the use of deionized water is most preferred.

In another embodiment, the solvent is an alcohol, such as for example ethanol.

On the other hand, in order to reduce surface tension, the inventors have shown that it was particularly advantageous to add to the liquid formulation a surfactant, preferably in an amount comprised between 0.05 and 2% by weight.

Thus, in a preferred embodiment, the formulation comprises:
a solvent; and
0-2% by weight of a surfactant of pharmaceutical quality, preferably in an amount comprised between 0.05 and 2% (by total weight of the solution).

An exemplary formulation is:
an aqueous solvent, comprising 0-15% of alcohol (by total volume of the solution); and
a surfactant in an amount comprised between 0.05 and 2% (by total weight of the solution).

The surfactant may be any surfactant compatible with pharmaceutical use, such as for example VOLPO N20.

Further, it may be preferred to dialyze the substance prior to its formulation.

The contained substance or formed by the liquid formulation (21) deposited on the patch may be any pharmaceutical, cosmetic, vaccinal and/or diagnostic substance (and/or synthetic analogs thereof), The substance (21) may be of biological nature and notably contain oligopeptides, biologically active and/or antigenic (poly)peptides or proteins, hormones, cytokines, immunoglobulins, allergens, growth factors, trophic factors, moisturizing compounds, vitamins, or chemical molecules. It may also contain drugs or active ingredients of various nature, either analogs of biological products or not, and non-exhaustively: nicotine, caffeine, morphine, hydromorphone HCl, fentanyl, apomorphine HCl, Scopolamine, chlorpheniramine, imiquimod, diphenhydramide, Lidocaine, Isotretinoin, Ketoprofen, Diclofenac, Leuprolide, Finasteride, etc. The substance may also be a combination of biological and non-biological compounds.

### Liquid Feeding Device

In a particular embodiment, a pumping device (14) is used for bringing the formulation (21) present in a tank (15), to the spray nozzle (11) with a controlled liquid flow rate. In a preferred embodiment, a syringe pump is used as a pumping device. Depending on the properties of the substance, the latter is generally taken up from the tank (15) at a temperature comprised between 4 and 60° C., preferably 20° C.

The liquid flow rate is adjusted in order to control the size of the formed droplets and to allow acceptable evaporation of the solvent, after or during deposition.

The rated flow of the formulation (21), for one nozzle (11), may for example be comprised between 0.01 and 100 millilitres/hour.

In a preferred embodiment, the rated flow for 1 nozzle of the formulation (21) is comprised between 0.01 and 10 milliLitres/hour, preferably between 0.01 and 1.5mL/hour, most preferentially between 0.1 and 1.5 mL/hour. The inventors have actually shown that with this flow rate it is possible to obtain droplets with an average size of less than 20 μm, preferably 5 μm, typically about 1 μm, ensuring formation of homogeneous deposits. When the substance is a polypeptide or a peptide, the flow rate is most particularly adjusted between 0.7 and 1.3 mL/hour.

In a preferred embodiment with several nozzles simultaneously, each nozzle is connected to a particular pump, the pumps being actuated simultaneously in order to produce an identical flow for the same duration.

On the other hand, the pump is equipped with a motor with which the direction of the pumping may be modified. Thus, the syringe is either filled without disassembling the latter by pumping the formulation via a container, or emptied by feeding the nozzles with the formulation.

### Gas Injection Device

In the ElectroSpray principle, an electric field at the surface of the liquid is required in order to be able to generate an aerosol of particles and to stabilize the EHDS method (in a stable mode). In air, at atmospheric pressure, this polarization of the liquid is at the origin of an electric field in the gas, which may induce ionization and discharge phenomena in the gas volume around the liquid, and thus destabilize liquid EHDS. Indeed, these pulse discharges are at the origin of changes in electric field over time at the surface of the liquid and thus in the average diameter and average charge of the produced droplets. In order to avoid these pulse discharge phenomena, while retaining the electric field required for forming the aerosol, it is possible to increase the dielectric permittivity of the gas by using an insulating gas (SF₆, CO₂, N₂O or any other gas or insulating gas mixture known to one skilled in the art).

All these solutions may be applied to the method of the invention in order to stabilize the production of patches according to the formulation to be sprayed and therefore according to the substance to be deposited.

According to a preferred embodiment, notably in the case of the use of an insulating gas, the device comprises a conduit (17) surrounding the free end of the nozzle (11) and intended for conveying gas. Advantageously, the gas is carbon dioxide.

The conduit (17) is then connected to a gas supply (18) and opens out at the free end of the nozzle (11).

The enclosure of the projection device may also be confined and air may be replaced with a more insulating gas.

### Properties of the Patch Support

The term of <<support>> as used in this document, designates the material or the surface area of the patch on which the substance contained in the formulation (21) is deposited by spraying. This support may be of various shapes and natures.

The support (31) should be conductive, at the surface or in the bulk, i.e. based on conductive material(s) or treated at the surface or in the bulk so as to be made conductive by any technique known to one skilled in the art. The support may thus comprise or consist of different biocompatible materials, such as for example polymeric material, doped polymer, polymer coated with a conductive layer on one or on both of the faces, metal, textile and/or biological material, etc.

In another embodiment, the support comprises at least one conductive face which is positioned facing the nozzle. A preferred support thus consists of an insulating layer, for example an insulating polymer (film, fiber, etc.) covered on at least one face, with a conductive layer.

The conductive layer(s) covering one or both of the faces of the support may be of inorganic nature (metal for example) or organic nature (for example comprising carbon, graphite or oxide(s)). The metal is preferably gold, silver, platinum or aluminium. The conductive layer(s) advantageously have a thickness comprised between 5-40 nm, preferably between 5-20 nm.

In the case of a conductive layer in graphite, graphite deposition on the support (31) may be carried out beforehand or on line, just before the step for electrohydrodynamically spraying the formulation (21). Graphite deposition may be carried out by projecting a neutral or charged aerosol or by impregnation by having the film pass in a graphite solution bath.

The formation of the conductive layer of the support before the spraying step, may further be achieved by metallization or depositing oxides. The oxide is preferably indium oxide doped with tin (ITO).

A plasma treatment may also be performed in order to promote La., adhesion to the deposit-support interface.

Thus, according to a particular embodiment, the invention lies in a method further comprising a step for treating the support before the spraying step consisting in a plasma treatment at low pressure or atmospheric pressure, and/or in metallization, and/or deposition of oxide and/or deposition of graphite.

In a preferred embodiment, the support is a support in polyethylene-terephthalate (PET) film covered with a thin conductive gold layer (15 nm). The resulting patch may further include an insulating dual-adhesive crown, for example in PE-PP foam.

According to a particular preferred embodiment, the support therefore includes at least one electrically conducting face, for example formed according to the method described above, and the aerosol (22) is projected on this electrically conducting face. Preferably, the support of the patch on which the substance is projected is essentially planar.

According to another particular embodiment, the support consists of an insulating polymer coated with a conductive layer and the electric field is formed by applying a potential difference between the spray nozzle (11) and the support (31) connected to the ground through at least one of the contacts (41, 44) directly connected to the ground and to the contact of the support (31) and positioned in contact with the conductive face of the support.

According to another preferred embodiment, when the support consists of a conductive material, the electric field is formed by applying a potential difference between the spray nozzle (11) and the support positioned between the nozzle and the counter-electrode (16).

The shape and the nature of the patch support may vary. Thus, although the support (31) illustrated in FIGS. 1, 2 and 3 is flat, other geometries may be contemplated. Notably supports comprising a depression forming a chamber, patches with a reservoir, rigid or semi-rigid supports, either planar or not, of circular, square, rectangular, oval shape, etc., depending on the needs.

The support applied in the method may be machined beforehand as a patch. In this case, the patch is directly used in the method of the invention.

In another embodiment, the support is coated beforehand with a substance according to the method of the invention, and then subsequently used in order to form a patch. In this case, the support (31) may for example appear as a film or roll on which the substance is projected. The patch intended for the final user will then subsequently be cut out from this film.

In this respect, as the conductive surface of the support preferably has to be perfectly connected to the ground for the whole duration of the deposition, and during the transfer from one patch to another, in order to allow flowing of the charges of the particles which are deposited and which accumulate on the support, in a particularly advantageous embodiment, the patch support appears as a roll-shape wide strip which is unwound gradually. The wide strip advantageously comprises:
the conductive support, for example as a film (for example PET coated with gold), and
a foam film including circular holes at regular intervals and adhered on the conductive film, the support area visible through each hole forming a patch deposition area.

The support film is wider than the foam film, so that each support area surrounded with foam is in electrical contact with the whole of the conductive surface (upper face) of the support film. This support film area will be connected, during its manufacturing, to the ground, via a conductive roll, itself connected to the ground. The patch is cut out (external cut) after deposition.

Deposition Method

For carrying out the method, the liquid formulation is provided to the nozzle, preferably under the mentioned formulation and flow rate conditions. The electric field is formed, causing formation of an aerosol, the droplets of which advantageously have an average size of less than about 5 μm. The particles which form on the support from the aerosol are collected on the support of the patch, which is then or simultaneously treated in order to evaporate any solvent residue and form a dry deposit. Thus, after and/or during the projection of the substance on the support (31), the possible residues of solvent in which said substance is dissolved, may be evaporated. Evaporation may be achieved passively, or by accelerated evaporation, for example by heating by convection, by irradiation (for example with ultraviolet or infrared radiation), by freeze-drying or circulation of dry gas. In a particular embodiment, drying of the support (31) is achieved by placing it in a flow of hot air.

In a preferred embodiment, the method of the invention further comprises a step for evaporating the solvent during and/or after deposition of the aerosol (22) so as to obtain a substance as dry residues. The evaporation step may be achieved by heating by convection, by irradiation, by freeze-drying and/or by circulation of dry gas.

As mentioned earlier, most of the deposition methods, such as depositions in dry forms, generally cause losses of active substance outside the area of interest. In this context, another major benefit of the invention lies in the focusing of the flow of active substance towards this area of interest (FIG. 6).

As schematized in FIG. 6, the surface covered by the flow of charged drops is controlled at two levels:
by the potential applied on the ring, and/or
by material delimitation of the deposition area, this delimitation may be achieved by the adhesive collar forming the peripheral portion of the patch (in particular for patches with a condensation compartment); this collar being electrically insulating.

In the latter case, the insulating collar, a constituent of the finished patch, specifically delimits an area in which the electric field lines end up and are focused. With this phenomenon, it is thereby possible to focus the flow of active substances, which follow the field lines, exclusively at the centre of the patch, avoiding any loss of active substance outside the area of interest and forming a perfectly localized deposit.

Another object of the invention relates to any patch obtained by the method described in the present application. The patch according to the invention consists of a support (31) on which a substance (21) has been deposited by electrospray, which is present as a dry deposit (33).

The patch is advantageously packaged so that the dry deposit (33) is insulated from the outside environment. Thus, as illustrated in FIG. 2, the patch (3) may comprise, in a particular embodiment, a peelable film (32) covering the powder (33) and the portion of the support (31) not covered by the powder (33). The peelable film (32) is intended to be removed after applying the patch (3) on the skin.

The invention is adapted to any type of patch, i.e. any device which may be applied on a skin area of a subject in order to put it into contact with a substance or to create a moisturizing area. These may be patches with passive, facilitated or mechanical diffusion, adhesive patches, bandages, plasters, cupules, or (trans)dermal patches.

The plasters consist of an adhesive mass or coating, containing one or more substances, one or more diluents, emollients and adhesives sprayed in a uniform layer on a suitable support. The adhesive mass is such that it softens and then adheres to the skin at the skin temperature. However, plasters retain the shape that was given to them during the manufacturing and adhere to the portions on which they have been applied. They appear as sheets of variable size, to be optionally cut out. They may be attached on an adhesive bandage and covered with perforated material in its centre intended to limit contact.

Medicinal bandages are intended to be applied on small skin lesions for local action and consist of an adhesive bandage on which a bandage material covered with a substance is attached in its centre.

Adhesive patches are intended to be applied on the skin in order to detect the sensitivity of an organ to a substance. These patches consist of an adhesive bandage with at its centre a plastic disc on which an adhesive mass containing the substance is placed. The adhesive mass further contains components such as gum arabic or gelatine and water.

Patches with passive, facilitated or mechanical diffusion, typically include a support on which the substance is deposited in dry form and if necessary, a device for facilitating cell permeation (application of electric pulses, ultrasound, micro-needles, etc.). Preferably a dry patch, notably of the occlusive type, notably an electrostatic patch is preferably used, as described in document WO 02/071950.

The patch according to the invention may notably be used in pharmaceutical, cosmetic, vaccinal and/or diagnostic applications.

In order to ensure preservation of the patch (3) in a packaging and to notably avoid alteration of the active ingredients of the deposited substance, and to preserve microbiological quality, the patch may be subject to additional treatment, such as for example pasteurization, ionization, and more generally, any treatment known to one skilled in the art.

Another object of the invention relates to a patch for applying a substance on the skin, said patch comprises said substance positioned on a support area of the patch, said support area being electrically conducting. As indicated above, the conductive support may be in conductive material(s) or treated at the surface or in the bulk in order to be conductive.

A more particular object of the invention relates to a patch for applying a substance on the skin, the patch comprising a support comprising an electrically conducting layer and an insulating layer, the electrically conducting layer being on the face of the support intended to be exposed to the skin, the substance being in dry form and immobilized on the conductive face of the support.

The substance advantageously appears in the form of microparticles. This may be any biological substance as described earlier, notably protein or peptide, for example antigens or allergens.

Moreover, according to a preferred embodiment, the periphery of the support is adapted in order to create, in contact with the skin, a sealed chamber containing said substance.

Other aspects and advantages of the present invention will become apparent upon reading the examples which follow, which should be considered as illustrated and non-limiting.

### EXAMPLES

### Example 1

### l Deposition of BSA on an Aluminized Polymeric Film

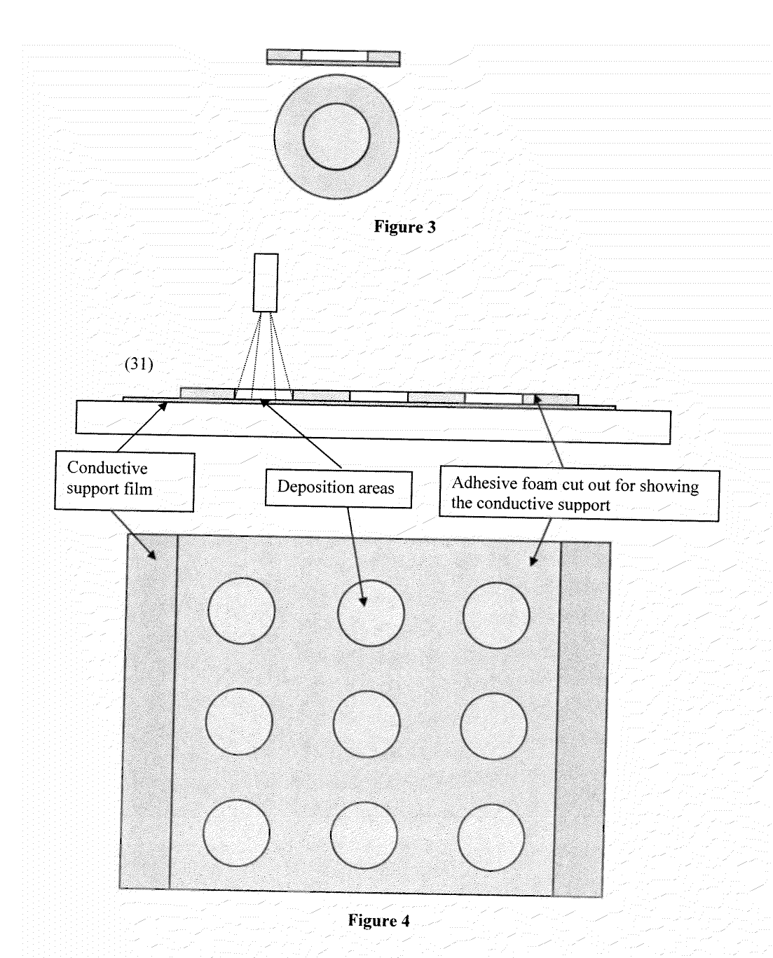

FIG. 1 illustrates an ElectroHydroDynamic Spray device (1) during the manufacturing of a patch (FIGS. 2 and 3) according to an embodiment of the method of the present invention.

In this particular embodiment, the spray device (1) comprises a nozzle (11) having an orifice for letting through a liquid, fed by a pumping device (14) which takes up a liquid formulation (21) from a tank (15). The liquid formulation (21) contains BSA (bovine serum albumin) dissolved in water. This formulation (21) is preferably provided to the spray nozzle (11) at a constant flow rate during spraying.

The counter-electrode (16) is positioned in the axis and at a distance from the nozzle (11). The counter-electrode (16) is connected to the ground.

The support (31) of a patch (3) is placed between the spray nozzle and the counter-electrode (16). This support (31) consists of a polyethylene polymer doped with carbon.

The spray device (1) further comprises a conduit (17) connected to a gas supply (18 and which surrounds the free end of the nozzle (11). The BSA was solubilized in low electric conductivity water (comprised between 10 and 100 μS/m ideally).

Deposition of BSA was achieved under stable conditions for a BSA concentration comprised between 0.1 and 5 mg/mL, liquid flow rates comprised between 0.1 and 2.5 mL/h, voltages comprised between 4 to 7 kV, a nozzle (11)/counter-electrode (16) distance from 0.5 to 1.5 cm, at atmospheric pressure, for a CO₂ flow rate comprised between 3 and 6 L/min and nozzles with outer and inner diameters respectively comprised between [0.11-0.60] mm and [0.006-0.1] mm.

Under these conditions of production, characterizations were made on the deposits:

i) The protein mass was first of all quantified by making assays with bicinchronic acid (BCA). These assays confirmed deposit of protein on the conductive supports in amounts comprised between 1 and 50 micrograms for BSA concentrations comprised between 0.1 and 5 mg/mL and a deposition time of one minute.

ii) The observations made by Scanning Electron Microscopy (SEM) then allowed verification of the homogeneous distribution of dry residues on the patch and non-degradation of the proteins by the method according to the invention was checked by means of electrophoresis gel which did not reveal any structural modification of the protein.

iii) Further, maintaining one of the main functions ensured by the BSA protein (antigen-antibody recognition) was validated by a radial immunodiffusion method.

These operating tests, shown in this example, have therefore demonstrated the fact that the manufacturing method of the invention allows a patch to be obtained which has a homogeneous distribution on the support, without alteration of the deposited substance.

### Example 2

### Patch with Conductive Support

In a preferred embodiment, the patch consists of:
a support in polyethylene-terephthalate (PET) film covered with a thin conductive gold layer (15 nm), and
a dual-adhesive insulating crown in PET foam (FIG. 3).

The patch on which a deposit is made by ElectroSpray, is provided with a conductive support, the conductive surface of the support should be perfectly connected to the ground or to a voltage generator and this for the whole duration of the deposition and during the transfer from one patch to another in order to allow flowing of the charges which are deposited at the same time as the substance particles which accumulate on the support. Knowing that the conductive surface is positioned facing the nozzle, for most of the time, it is not possible to directly carry out this grounding operation by simply contacting the support on a table itself connected to the ground. The solution found consists of having the material forming the patches appear as a wide roll strip which is unwound gradually.

The wide strip comprises:
the conductive support as a film (for example PET coated with gold), and
a foam film including circular holes at regular intervals and adhered on the conductive film, the support area visible for each support forming a deposition area of a patch (FIG. 4).
   The support film is wider than the foam film so that each support area surrounded with foam is in electric contact with the whole of the conductive surface (upper face) of the support film. This support film area will be connected during manufacturing to the ground, via a conductive roll to the ground. The patch is cut out (external cutting) after deposition.

### Example 3

### Physical Characters of the Substance Deposit

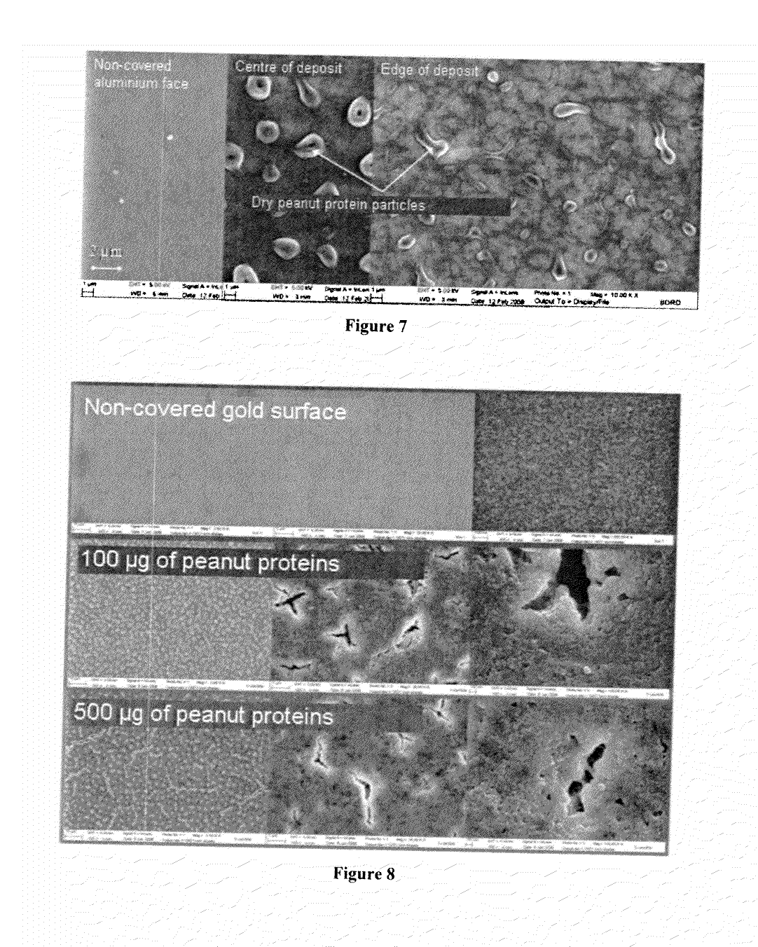

In certain cases, the evaporation of the solvent(s) during the transit time of the drops suspended in the gas is sufficient so that the substance appears on the patch as dry distinct and well-individualized particles (FIG. 7). The size of these particles facilitates their adhesion onto the support in particular under the action of Van der Waals forces.

In other cases, the flow rate of particles and the nature of the solvent are such that the peanut proteins are deposited on the support in humid form and may then aggregate with each other. Visually, these deposits appear as a homogeneous layer. However, surprisingly, dissolution of this layer is extremely easy, which makes the substance extremely available, as demonstrated by the tests made with deposits produced by the inventors with BSA or peanut proteins. Thus, it is sufficient to wipe a barely moist cloth on the support in order to remove the quasi-totality of the deposit. Patch-tests made by this technique and deposited on patients allergic to peanut, have shown the rapidity of action of the patch, due to the great availability of the substance. Photographs taken with a Scanning Electron Microscope (SEM) enable identification of the morphology of these deposits and of their features:

A) the multi-layer structure of the deposit, and

B) the presence of micro-holes or cracks in the deposit (FIG. 8).

Both of these particularities are probably at the origin of this capability of being rapidly solubilized: diffusion of moisture is promoted both by the superposition of the layers and by the presence of these micro-holes.

To summarize, the ElectroSpray method applied to so-called <<dry patches>> produces deposits, for which the strong porosity that photographs taken by electron microscopy seem to show, is an adjuvant factor for administration of these substances.

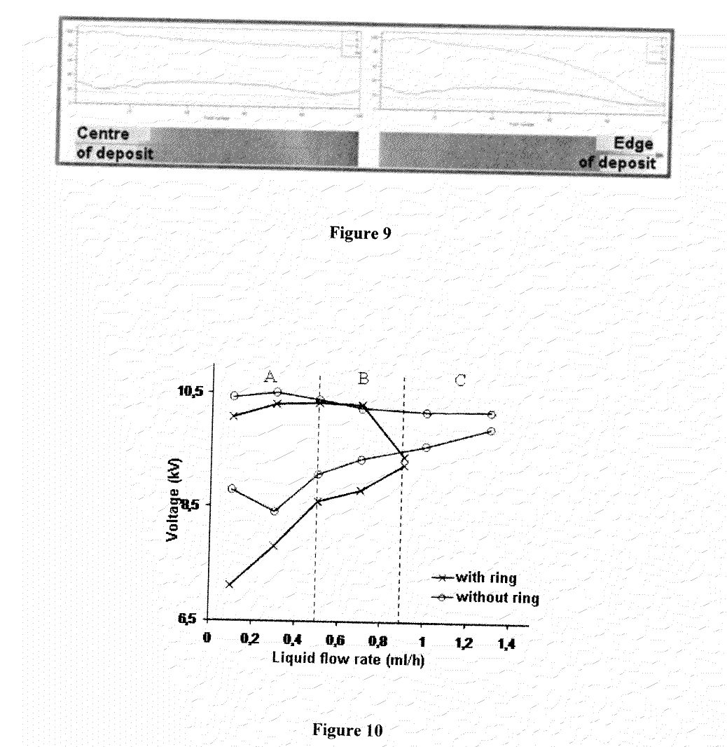

Another interesting property of the method appears upon examining analyses of the elementary composition of peanut protein deposits by EDS. These analyses demonstrate a slight decrease in the amount of carbon (red curve in FIG. 9), characteristic of the amount of organic material and therefore of deposited proteins, when moving gradually away from the centre of the deposit.

The deposit is therefore particularly homogenous, an important property as it is known that diffusion of the substance of the patch towards the skin is carried proportionally to the concentration of the substance and perpendicularly to the surface of the skin.

### Example 4

### Deposit of Peanut Protein Extract on a Patch with a Polymeric Support (PET) Covered with a Gold Layer

For this deposition, the nozzle (11) is fed with a peanut liquid formulation (21) to be deposited, at a liquid flow rate advantageously equal to 0.7 mL/h. The nozzle (11) is placed at 18 mm from a wide strip of preformed patches (FIG. 4) mainly consisting of a PET/GOLD support (conductive at the surface) and of a dual-adhesive (insulating) foam washer. The nozzle (11) and the liquid (21) containing the peanut protein extract are polarized to a high voltage by a high voltage power supply (14), preferentially to 9-9,5 kV. In order to have the electric charges flow over time and to ensure stability of the method, the conductive face of the support is connected to the ground, thereby positioning all the supports of preformed patches connected to the ground.

In order to allow industrialization of such a method for manufacturing patches, i.e. in order to increase the robustness of the method during the manufacturing of the patches, a shielding ring (12) is positioned at 5 mm from the nozzle (11). In a preferential embodiment, it is polarized to 2.2 kV.

Under these conditions, deposition of a peanut protein extract from one patch to the other (which notably involves the passing of the flow of active substances over areas which are successively conducting and insulating) is made possible without interrupting the method for the reasons mentioned earlier.

Two embodiments have been tested for illustrating this second example: with or without a ring connected to the ground.

The support used is polymeric PET film (thickness of 23 μm) covered with a thin gold layer (15 nm).

The peanut formulation which may be sprayed with the ElectroSpray method is obtained by dissolving the peanut protein extract in a mixture of milliQ water, ethanol (99.9%) and a non-ionic surfactant (Volpo N20).

For this given peanut formulation, the operating ranges obtained in the nozzle-support and nozzle-ring-support configuration have been plotted. In order to allow them to be compared, these ranges have been defined with nozzle-support (without any ring) and nozzle-ring distances equal to 20 mm. In the latter case, the ring is itself positioned at 20 mm from the plane in order to avoid any influence of the latter on EHD equilibrium (FIG. 10).

As indicated in FIG. 10, three distinct areas may be defined:
A area: Succession of the drop production modes is standard: dropwise (GAG)=>intermittent cone-jet=>stable mode=>multi-cone jet.
B area: The multi-cone jet mode no longer exists in a nozzle-ring-plane configuration. Beyond the maximum voltage of the stable mode, the liquid cone remains centred relatively to the axis of the nozzle but pulse discharges perturb the production mode.
C area: Stability of the method in the nozzle-ring-plane configuration is no longer possible because of pulse discharges.

These differences observed in the B and C areas may be ascribed to modification of the electric field lines between the nozzle and the relevant counter-electrode (ring or plane connected to ground).

### Example 5

### Deposition of LHRH by ElectroSpray

A third type of protein was tested in order to confirm the feasibility of depositing an active ingredient by EHDS, for making patches in particular.

Table 1 shows the experimental conditions tested within the scope of depositing LHRH:

TABLE 1

| Support | Prototype Gen1. |
|---|---|
| PET film (23 μm) with | Nozzle-film configuration |
| gold coating (15 nm) | (without any shielding ring) |
| Multi-layer PP, | U_{nozzle} ~ 8-9 kV |
| aluminium coating | D_{ext/int nozzle} = 6/0.3 mm |
| | D_{nozzle-plane} ~ 3 cm |

A 7 mg/mL solution of LHRH diluted in ethanol (99.9%) was tested. The conductivity and surface tension are equal to 5,400 μS/m and 21.8 mN/m, respectively. By using an outer nozzle diameter of 6 mm, placed strictly perpendicularly to the surface of the film, a stable mode may be obtained, comparable to the one obtained with peanut.

Obtaining an instantaneous dry deposit was achieved on films metallized with aluminium or gold.

The aspect of the deposit is similar to that of the peanut deposits. The diameter comprised between 3 and 3.3 cm may be explained by the inter-electrode distance which is about 30% larger than what is usually used for peanut.

1. A method for manufacturing a patch intended for skin application of a substance, wherein the patch includes a conductive support, the improvement comprising depositing a liquid formulation of the substance on the support of the patch by electrohydrodynamic spraying.

2. The method of claim 1, wherein the depositing the liquid formulation of the substance comprises:
a) placing the support at a distance from a spray nozzle;
b) providing the liquid formulation containing the substance to the spray nozzle to form an aerosol;
c) applying an electric field to the aerosol; and
d) collecting on the support particles from the aerosol.

3. The method of claim 1, wherein the substance is dissolved in a biocompatible solvent.

4. The method of claim 3, wherein the substance comprises a protein and the solvent comprises 0-15% alcohol by volume and 0-2% surfactant by weight.

5. The method of claim 3, wherein the solvent is an alcohol.

6. The method of claim 2, wherein the substance in aerosol form comprises droplets having an average diameter of less than or equal to 20 μm.

7. The method of claim 2, wherein the electric field is formed by applying a potential difference between the spray nozzle and the support, wherein the support is coupled to a ground.

8. The method of claim 2, wherein the electric field is formed by applying a potential difference between the spray nozzle and a counter-electrode, the counter-electrode comprising a plate with holes.

9. The method of claim 2, wherein the electric field is formed by applying a potential difference between the spray nozzle and a contact coupled to the ground and to a contact coupled to the support.

10. The method of claim 2, wherein the formation of the aerosol is carried in ambient air or in a gas atmosphere other than air gas.

11. The method of claim 1, wherein the substance comprises a pharmaceutical, cosmetic, vaccinal or diagnostic substance and comprises a polypeptide, a protein, or a chemical molecule.

12. The method of claim 5, wherein the alcohol comprises ethanol.

13. The method of claim 2, wherein providing the liquid formulation comprises providing the substance to the spray nozzle at a constant flow rate, preferably of less than 1.5 mL/hour.

14. The method of claim 1, wherein the support comprises conductive material.

15. The method of claim 1, wherein the support comprises a conductive face positioned facing the nozzle.

16. The method of claim 1, wherein the support comprises a doped polymer, a biocompatible metal, or a polymer coated with a conductive layer on one or both faces of the support.

17. The method of claim 16, wherein the conductive layer of comprises metal, carbon, graphite, or oxides.

18. The method of claim 17, wherein the metal comprises gold, silver, platinum, or aluminium.

19. The method of claim 17, wherein the oxide comprises indium oxide doped with tin (ITO).

20. The method of claim 1, further comprising treating the support, before the electrohydrodynamic spraying, using plasma treatment at low pressure or at atmospheric pressure, metallization, oxide deposition, or graphite deposition.

21. The method of claim 1, wherein the substance is coupled to an electrically conducting face of the support.

22. The method of claim 2, wherein the support comprises an insulating polymer coated with a conductive layer and the electric field is formed by applying a potential difference between the spray nozzle and the support connected to a ground through a contact directly connected to the ground.

23. The method of claim 1, wherein the support is essentially planar.

24. The method of claim 3, further comprising means for evaporating the solvent during and after the electrohydrodynamic spraying.

25. The method of claim 24, wherein the evaporation means is achieved by heating, by convection, by irradiation, by freeze-drying, or by circulation of dry gas.

26. The method of claim 1, further comprising packaging the support to insulate the substance after the electrohydrodynamic spraying.

27. The method of claim 1, further comprising covering the support with a peelable film after the electrohydrodynamic spraying.

28. A patch for skin application of a substance, obtained by the method of claim 1.

29. A patch for skin application of a substance, said patch comprising said substance positioned on a supporting area of the patch, said supporting area being electrically conducting.

30. A patch for skin application of a substance, the patch comprising a support comprising an electrically conducting layer and an insulating layer, the electrically conducting layer being on the face of the support intended to be exposed to this skin, the substance being in dry form and immobilized on the face of the support.
